(19) United States
(12) Patent Application Publication
(10) Pub. No.: US 2025/0384357 A1
(43) Pub. Date: Dec. 18, 2025

(54) System and display for asset availability

(71) Applicant: Elemental Machines, Inc., Cambridge, MA (US)

(72) Inventors: Casey Peters, Caldwell, NJ (US); Sridhar Iyengar, Salem, NH (US); Timothy Golnik, Bellevue, WA (US)

(73) Assignee: Elemental Machines, Inc., Cambridge, MA (US)

(21) Appl. No.: 18/867,506

(22) PCT Filed: Jun. 7, 2023
(86) PCT No.: PCT/US2023/024689

§ 371 Date: Nov. 20, 2024

Related U.S. Application Data
(60) Provisional application No. 63/351,097, filed on Jun. 10, 2022.

(51) Int. Cl. G06Q 10/02

(52) U.S. Cl. CPC G06Q 10/02

(57) ABSTRACT

A system for determining availability of an asset is provided. The system includes a sensor positioned to determine an electrical property of the asset and a module and/or programmed circuitry having hardware and instructions including those for performing the following: (i) obtaining a data set comprising readings from the sensor over a period of time; (ii) determining ranges of sensor readings by performing statistical analysis of the data set obtained in step (i); (iii) assigning asset operating state values to the ranges of sensor readings determined in step (ii); (iv) obtaining a further reading from the sensor; and (v) determining the operating state of the asset by comparing the further reading from the sensor obtained in step (iv) to the asset operating state values assigned in step (iii), thereby determining the availability of the asset.

### BACKGROUND OF THE INVENTION

Many organizations have shared physical assets. This is common in industries that work with physical materials, such as biotechnology companies, pharmaceutical companies, materials science companies, testing labs, factories, quality control labs, research and development labs, clinical labs, etc. Many of these have facilities with shared instruments, equipment, and/or machines (collectively called “assets”).

When such organizations grow in size, they face the challenge of having many people requiring use of the same set of assets. Furthermore, if the physical layout of the facility is large, it becomes a burden for users to travel to a particular room, lab or work area to determine if an asset is free to use.

Some companies have developed software programs to address this challenge by developing reservation systems. There are several reservation systems that allow users to reserve time on assets, such as those provided by:
Elemental Machines: https://elementalmachines.com/calendar
BookitLab: https://bookit-lab.com/equipment-scheduling/
Cluster Market: https://www.clustermarket.com/de-campaign/booking
Lab Archives: https://www.labarchives.com/scheduler/

However, even when there is a reservation system, it is not uncommon for someone to not use the reservation system to reserve the use of an asset, but instead to simply use the asset without reserving it. Also, it is not uncommon for someone to reserve time to use an asset and then simply neglect to actually use it during the allotted time, thereby reducing the availability of that asset for others in the organization.

It is therefore important and useful to know when an asset is actually free to be used or if it is actually currently in use.

### BRIEF SUMMARY OF THE INVENTION

The present invention provides solutions to the problems noted above by providing a system for determining availability of an asset and associated displays, files, and methods. The system includes a sensor positioned to determine an electrical property of an asset and a module and/or programmed circuitry comprising hardware and instructions for performing steps of the herein-described methods to determine availability of the asset. The steps includes: (i) obtaining a data set comprising readings from the sensor over a period of time; (ii) determining ranges of sensor readings by performing statistical analysis of the data set obtained in step (i), (iii) assigning asset operating state values to the ranges of sensor readings determined in step (ii); (iv) obtaining a further reading from the sensor; and (v) determining the operating state of the asset by comparing the further reading from the sensor obtained in step (iv) to the asset operating state values assigned in step (iii), thereby determining the availability of the asset.

### BRIEF DESCRIPTION OF THE DRAWING

FIG. 1 shows readings from sensors associated with different laboratory and/or manufacturing equipment.

FIG. 2 shows readings from sensors associated with laboratory and/or manufacturing equipment from the same manufacturer.

FIG. 3 shows a data set containing readings from one or more sensors.

FIG. 4 shows an embodiment of a visual display (e.g. a dashboard) from an asset management/reservation system.

FIG. 5 shows an embodiment of a visual display (e.g. a dashboard) from an asset management/reservation system.

FIG. 6 shows an embodiment of a visual display (e.g. a dashboard) from an asset management/reservation system.

FIG. 7 shows an embodiment of a visual display (e.g. a dashboard) from an asset management/reservation system.

FIG. 8 shows an embodiment of a visual display (e.g. a dashboard) from an asset management/reservation system.

FIG. 9 shows an embodiment of a visual display (e.g. a dashboard) from an asset management/reservation system.

FIG. 10 shows an embodiment of a visual display (e.g. a dashboard) from an asset management/reservation system.

### DETAILED DESCRIPTION OF THE INVENTION

The present invention solves the above-described problems and provides inter alia a system for determining and informing a user if an asset is currently in use or if it is free to use and related files, methods, and displays.

One major challenge in determining whether or not an asset is in use or not in approximately real time is that there are many different types of assets by many different manufacturers, with many different models. Knowing when an asset is in use or not is not well defined. Furthermore, asset manufacturers rarely provide a means for electronically identifying if an asset is in use or not. For example, assets do not commonly broadcast a message (e.g. via wireless, ethernet, or other communication means) indicating whether they are currently in use or not. Notable exceptions include assets that may be hazardous when in use, and so these assets sometimes have visible or audible indicators to let users know that it is in use (for example, flashing lights or an audible alarm).

The challenge of determining whether an asset is in use or not is similar to the challenge of knowing when a conference room is in use or free to be used. In a conference room, it is possible to employ sensors to determine occupancy, for example like the ones listed here:
Adappt Intelligence: https://adappt.com/elementor-9256/
https://www.rayzeek.com/blog/everything-about-occupancy-and-vacancy-sensors

Also, motion sensors (e.g. Passive Infrared sensors) like those used in home security systems can also detect if a room is occupied by detecting the motion of its occupants.

However, there is no simple analogous system for determining asset usage or patterns thereof. One commonly used method of determining usage metrics is by directly measuring the power and/or current that it draws. In general, the assets that are of interest are those that draw power from a wall circuit (also called mains power). These assets generally have a power cord that is plugged either directly or indirectly (e.g. via a transformer, multi-plug, extension cord, or a travel adapter) into a power socket in a wall.

Various manufacturers offer “smart plugs” that can be plugged into a wall socket, into which the asset can then be subsequently plugged in. These smart plug devices generally measure the current that is passed through them (from wall power to the connected asset), and have some means of communication (usually wireless, e.g. WIFI, RF, Bluetooth, BLE, ANT, ZigBee, etc.) to transmit a measure of the power and/or current drawn by the asset. Examples of “smart plugs” include:
WEMO made by Belkin
Amazon Smart Plug
WattIQ: https://wattiq.io/

Split core current transformers (SCCTs) are another type of sensor system for measuring current passing through a conductor. SCCTs can be clamped around a supply line of an electrical load to provide an indication of how much current is passing through it. SCCTs work by acting as an inductor and responding to the magnetic field around a current-carrying conductor. By reading the amount of current being produced by the coil, SCCTs can calculate how much current is passing through the conductor.

Another way to determine a measure of the current flowing in a power cords by using Hall Effect sensors, such as the system described in US Patent Application Ser. No. PCT/US2021/018366, which is incorporated herein by reference for all purposes.

One ordinarily skilled in the art will recognize that power and current are related by the voltage, so for the sake of simplicity, we shall refer to all ways of determining a measure of the power and/or the current as “current sensing” and a device that determines a measure related to the current and/or power as a “Current Sensor”.

Even by sensing the current that an asset uses, it is still difficult to determine its use state. This is because many assets have current patterns that do not simply switch between two states indicative of being in use or not being in use. Furthermore, the current patterns for different types of assets (and even the same type of asset from different manufacturers, or even different models of the same asset type from the same manufacturer), can be different. Also, if the asset has different operating states (or different operations that it does), the current pattern can be different.

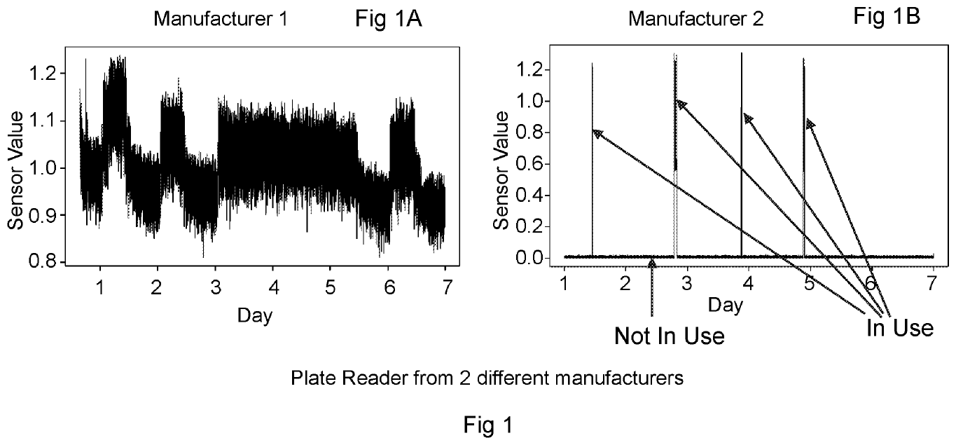

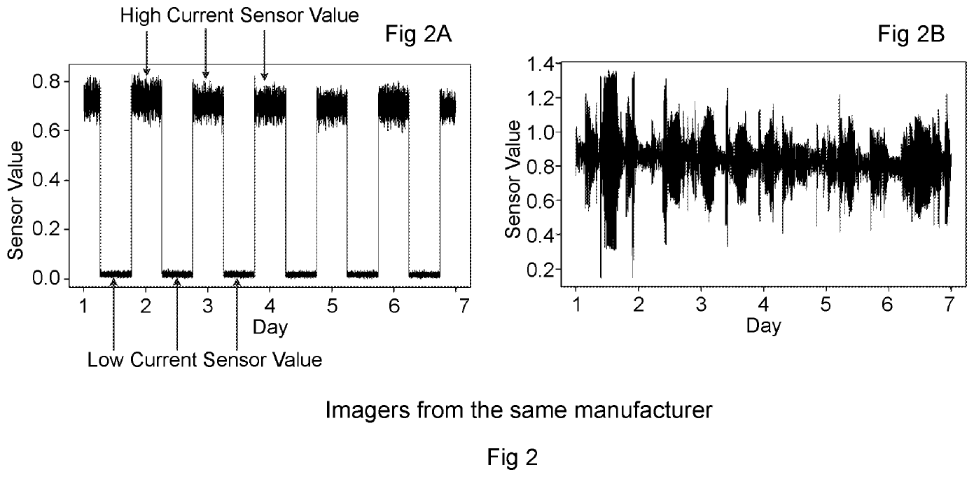

FIGS. 1 and 2 show this variability:
FIG. 1A and FIG. 1B both are plate readers, but they are made by different manufacturers. In FIG. 1B, it is clear that the spikes in the sensor value (that is, the Current Sensor) are indicative of when the plate reader asset is in use. However, in FIG. 1A, it is not as clear if or when the asset is in use since the current sensor value is not a clear spike up.
FIGS. 2A and 2B are both Imagers from the same manufacturer (Formulatrix), but they show very different Current Sensor readings. In FIG. 2A, there is a relatively periodic pattern of high current readings and low current readings. In FIG. 2B, there is no clear discernible pattern that would indicate that the asset is in use or not.

Thus, these examples show the difficulty in determining whether an asset is in use or not. Simply measuring the current that the asset draws is not sufficient as there are many different current patterns that can manifest, even across the same manufacturer or asset types.

As mentioned above, measuring the power and/or current and/or voltage that an asset uses is not new. However, these types of smart plugs generally just measure the total current and/or power that an asset uses and reports that to the user in a convenient form (usually a graph indicating total or average power and/or current for a given period of time).

This level of analysis is sufficient for an organization that is primarily interested in determining power consumption of their assets for economic and financial analysis purposes. However, for users to know whether an asset is free to use or if it is currently being used, further analysis of the current signal has been found to be useful.

As mentioned above, a challenge is that there are myriad different assets by many different manufacturers, and as seen in FIGS. 1 and 2, the same type of asset can have very different current signals. It is not economically possible to identify and model the specific current signal patterns for each and every asset when operated under each and every condition. Thus, there needs to be a way to automatically determine and learn over time what constitutes a current signal pattern that indicates that an asset is in use or that it is free.

One way to achieve this is to couple a Current Sensor to each asset of interest. Since each asset has an associated Current Sensor, it is also helpful to know whether the Current Sensor is functioning and transmitting the information that it is measuring. Thus, it is helpful to determine if the Current Sensor itself has somehow stopped functioning and/or is offline (“Sensor Offline” state).

Thus an asset and its associated Current Sensor can broadly be described to be in a few basic states, for example:
The asset is off (“Off” state)
The asset is on but is not being used (“Idle” state)
The asset is in use (“In Use” state)
Current Sensor is offline (“Indefinite-Sensor Offline” state)

There may be additional states that are useful to determine, like if an asset has different modes of operation. Knowing this may be useful since it provides additional context to perhaps how long that asset may be in use. For example:
High speed centrifuge vs low speed (the high speed may be a shorter use period)

A challenge with creating a robust system for such classification of states is that the actual true state of the asset is often unknown and not recorded. In machine learning, the true state is often called the “ground truth”. If the true state of the asset were known during a window of time, then the Current Sensor readings during that window can be associated with the true state and well-known supervised learning methods can be used for training a machine learning system (for example using a Neural Network, Regression or a Decision Tree, to name a few).¹ ¹ Supervised learning (SL) is the machine learning task of learning a function that maps an input to an output based on example input-output pairs. It infers a function from labeled training data consisting of a set of training examples. In supervised learning, each example is a pair consisting of an input object (typically a vector) and a desired output value (also called the supervisory signal). A supervised learning algorithm analyzes the training data and produces an inferred function, which can be used for mapping new examples. An optimal scenario will allow for the algorithm to correctly determine the class labels for unseen instances. This requires the learning algorithm to generalize from the training data to unseen situations in a “reasonable” way (see inductive bias). This statistical quality of an algorithm is measured through the so-called generalization error. See for example https://en.wikipedia.org/wiki/Supervised_learning.

A challenge, therefore, is to estimate and/or infer ground truth somehow. In preferred embodiments, the present invention provides various methods for estimating and/or inferring ground truth so that a classifier can more correctly determine the usage state of an asset.

### Clustering:

The general premise of clustering methods known in the art is to compare multiple values or data points and determine if they fall into groups or clusters. There are many different methods known in the art to determine if a particular data point or set of data points belong in a cluster, such as DBSCAN or k-means clustering.

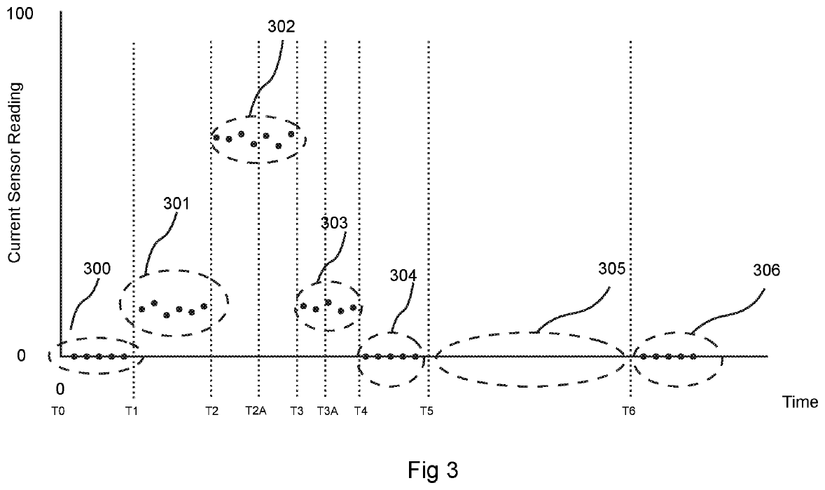

One method of clustering the data measured by a Current Sensor is to compare the readings directly. It is generally the case that the current that an asset uses in the In Use state is greater than the current that it uses in the Idle state, and furthermore, it will generally use more current while in the Idle state than when it is in the Off state. Thus, when the Current Sensor measures the current of the asset, the measured values may be grouped into different categories based on their values (such as maximum, minimum or mean values) or based upon statistical analysis in the time domain such as standard deviation or signal noise-to-noise ratio or the frequency domain such as resonant frequency or a set of FFT coefficients. FIG. 3 illustrates this.

In FIG. 3, a Current Sensor's reading as sampled over a period of time is shown. There are several groups of values that naturally group together:
A group of data points 300 between T0 and approximately T1 is seen to have virtually no Current Sensor reading and therefore is effectively zero. This can be assumed to be the Off state.
A group of data points 301 is seen to have low but non-zero Current Sensor reading. This can be assumed to be the Idle state since some current is being used by the asset, implying it is not completely off. Furthermore, this implies that a user has turned on the asset into its Idle state at approximately time T1
A group of data points 302 is seen to have high Current Sensor reading. This can be assumed to be the In Use state since a relatively higher current is being used by the asset, implying that it is performing some action that requires higher current usage than in the idle state. Thus, this implies that a user has actively started to use the asset at approximately time T2.
A group of data points 303 is seen to have low but non-zero Current Sensor reading. This can be assumed to be the Idle state since some current is being used by the asset, and further implies that the user has finished using the asset and it has now returned to its Idle state at approximately time T3.
A group of data points 304 is seen to have virtually no Current Sensor reading and therefore is effectively zero. This can be assumed to be the Off state, and furthermore, this implies that the user has turned off the asset completely at approximately time T4.
There are no data points 305 recorded between approximately time T5 and time T6. This implies the Current Sensor is in Offline state.
A group of data points 306 is seen to have virtually no Current Sensor reading and therefore is effectively zero. This can be assumed to be the Off state, and furthermore, this implies that the Current Sensor has come back online and exited the Sensor Offline state at approximately time T6.

Another method to define a boundary between each usage state is shown in FIG. 11. The readings of FIG. 3 may be analyzed via a histogram method as shown in FIG. 11:
There is a relatively large number of readings clustered around a Current Sensor reading of 0 as shown in the distribution 1100. This can be assumed to be the Off state.
There is another cluster of readings clustered around a Current Sensor reading of 15 as shown in the distribution 1101. This can be assumed to be the Idle state.
There is another cluster of readings clustered around a Current Sensor reading of 60 as shown in the distribution 1102. This can be assumed to be the In Use state.

A boundary threshold B1 1103 can be defined between the first cluster 1100 and the second cluster 1101. A boundary threshold B2 1104 can be defined between the second cluster 1101 and the third cluster 1102. Thus when new Current Sensor readings are taken, the system can classify the reading based on where the reading lies:
A reading below B1 can be identified as Off state
A reading between B1 and B2 can be identified as Idle state
A reading above B2 can be identified as In Use state

Thus, these are example methods of estimating and/or inferring ground truth from relative levels of the Current Sensor readings over time. The Current Sensor reading may be given as an absolute measurement or in relative terms of the full scale reading the Current Sensor is capable of.

The state of the asset in each of these time windows can now be determined and conveyed to a user in several ways, including:
Changing the color associated with an asset in a graphical user interface to be indicative of the state of the asset. For example:
   Green can be used to indicate Idle state
   Red can be used to indicate In Use state
   Gray can be used to indicate Off state
   Black can be used to indicate Sensor Offline state
Displaying a human readable message indicative of the state of the asset
Displaying an icon or other graphical image indicative of the state of the asset
Sending a notification including:
   Sending an email
   Sending a text message
   Sending a SMS message
   Sending a voice message or making a voice call
   Sending a message via an API to another software program

A user can also set up a notification system that sends a notification when the asset becomes available. For example, the user may instruct the system to “notify when free”. Preferably, the asset would have switched from an In Use state to an Idle state or Off state for a predefined duration of time, such as 5 minutes, 10 minutes, 15 minutes, 30 minutes, or one hour. This can ensure that there is enough buffer time to allow the previous user to complete any tasks associated with ending the use of the asset (such as cleaning, or removing a sample, etc).

Furthermore, the duration that an asset spends in each state can be computed by the difference in times when an asset changes its state. For example for the asset measured in FIG. 3:
The asset spends approximately a duration of time calculated by T1−T0 in the Off state
The asset spends approximately a duration of time calculated by T2−T1 in the Idle state
The asset spends approximately a duration of time calculated by T3−T2 in the In Use state
The asset spends approximately a duration of time calculated by T4−T3 in the Idle state
The asset spends approximately a duration of time calculated by T5−T4 in the Off state
The Current Sensor spends approximately a duration of time calculated by T6−T5 in the Sensor Offline state

In addition to the asset's current state, the system of the invention can inform a user or other interested party as to the duration of time that the asset has been in its current state of usage. For example:
At time T2A, the system can inform that the asset is currently in the In Use state and that it has been in its current In Use state for approximately a duration of time calculated by T2A−T2.
At time T3A, the system can inform that the asset is currently in the Idle state and that it has been in its current Idle state for approximately a duration of time calculated by T3A−T3.

In this manner, a user can gain insight not only into an asset's current state of use, but also how long it has been in that state. This is useful when the user may know some additional information about the asset of interest. For example, it is possible that a certain asset is programmed to run an assay, and the user knows that such an assay generally takes between 2-3 hours. Knowing that the asset is, say, 2.5 hours in the In Use state can give the user the insight that the asset might be freeing up for use in approximately 30 more minutes.

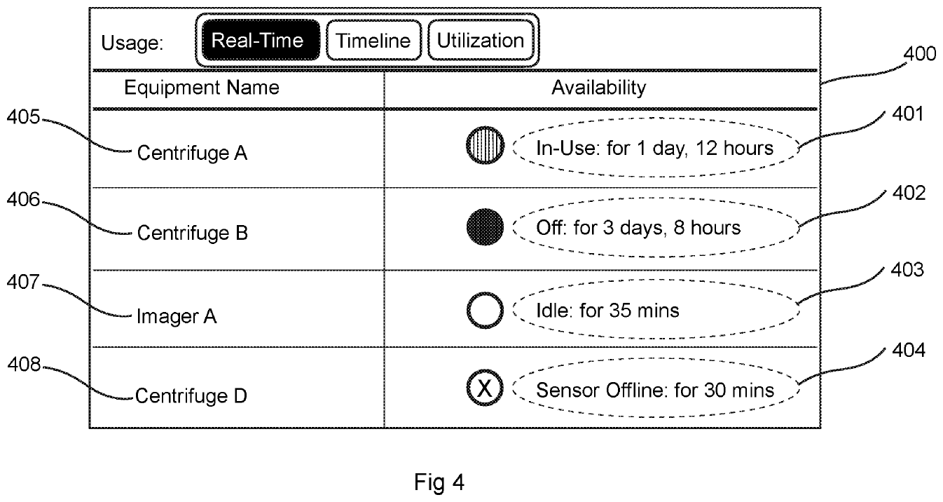

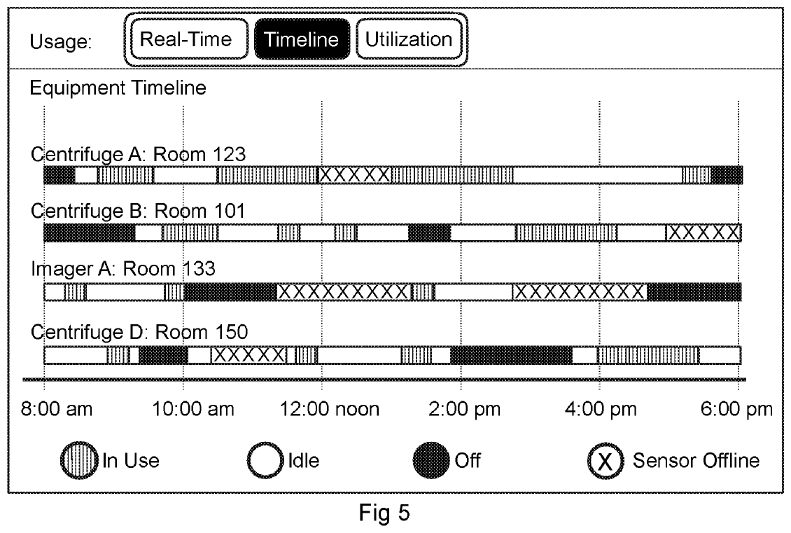

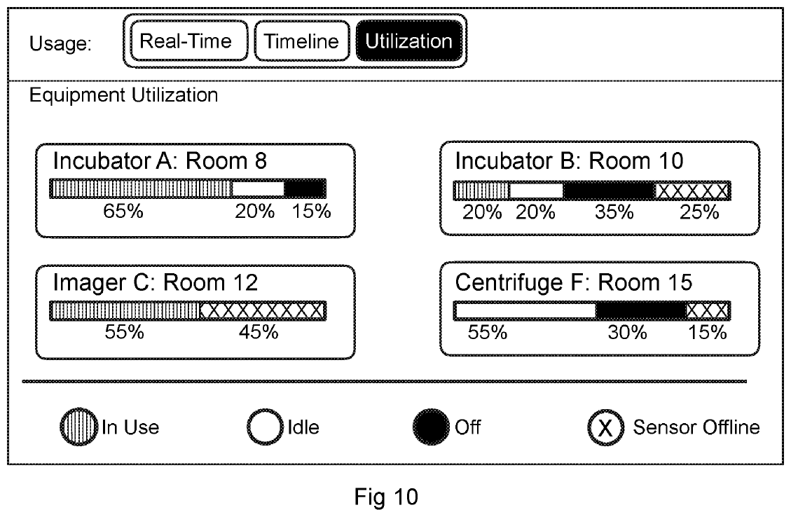

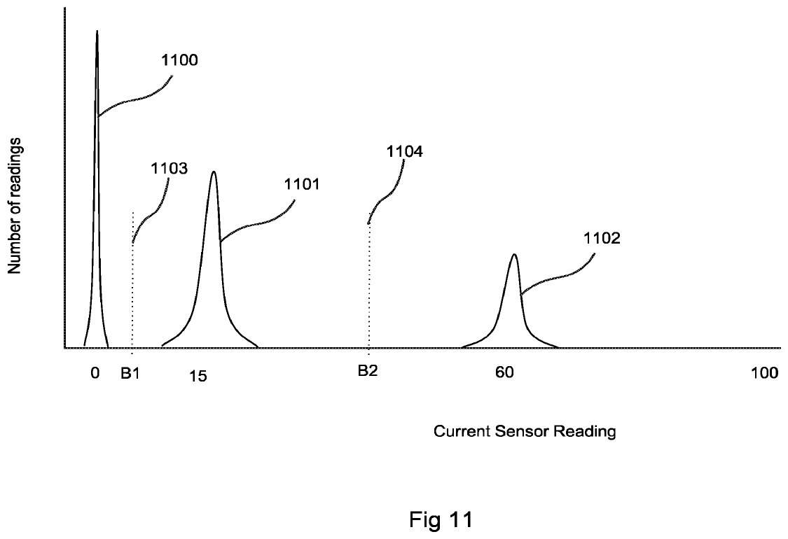

FIGS. 4-6 show examples of a graphical user interface displaying usage state information in different ways:
FIG. 4 shows a graphical user interface 400 that lists several assets in the first column along with the type of asset it is and where it is located. Additionally for each asset, the improved system of the invention shows the current usage state and duration that each asset has been in that usage state:
   Centrifuge 405 is shown to be currently in the In Use state and has been in this state for 1 day and 12 hours as indicated by colored dot and text content 401
   Centrifuge 406 is shown to be currently in the Off state and has been in this state for 1 day and 12 hours as indicated by colored dot and text content 402
   Centrifuge 407 is shown to be currently in the Idle state and has been in this state for 35 minutes as indicated by colored dot and text content 403
   The Current Sensor associated with Centrifuge 408 is shown to be currently in the Sensor Offline state and has been in this state for 30 minutes as indicated by colored dot and text content 404
FIG. 5 shows another representation of the usage state of assets. In this view, the usage state of each asset is shown in a horizontal timeline with windows of time color-coded for the different states being determined.
FIG. 10 shows a consolidated view where the amount of time each asset has spent in each different state is represented as a percent. This view is helpful for those involved in purchasing decisions to know whether an asset is being used more or less than other assets of the same category. They can then choose to purchase more of a particular type of asset if they deem that an asset is being overused. Alternatively, then can choose to retire an asset if that asset is deemed to be under-utilized. Also, for under-utilized assets, the asset manager may choose to cancel a support contract since not many users are using that particular asset.

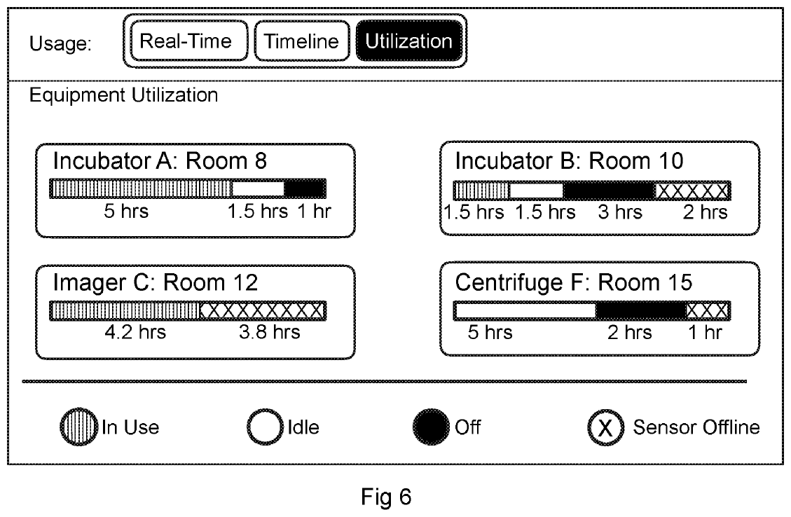

FIG. 6 shows the same data as in FIG. 10 but expressed as absolute time.

One ordinarily skilled in the art will recognize that additional usage states based on clustering current levels may be possible to identify. An asset may be configured to operate in different states where the current usage may be different. For example:
A centrifuge may be used at a low speed (for example 5000 RPM) or at a high speed (for example 10,000 RPM). A higher RPM speed would be expected to use more power/current. Thus in this example, There may be the following usage states that can be identified and reported:
   “Sensor Offline”
   “Off”
   “Idle”
   “In Use-slow speed” which corresponds to the centrifuge being operated at a slow speed
   “In Use-high speed” which corresponds to the centrifuge being operated at a high speed

It is also possible to have more complex algorithms and models of the current signal to find specific patterns that can give more insight into different operating states of an asset, such as the approach developed by sense .com. However, a major disadvantage to their approach is that they must sample at much higher frequencies to capture intricate details of the current signal and also employ much higher levels of computation. Both of these requirements drive up the costs associated with constructing and operating a Current Sensor and the associated algorithms. Furthermore, if the Current Sensor itself were to be powered by batteries, then higher levels of sampling and computation would decrease the battery life. Thus, there is a need for a way to determine the useful states of an asset and/or machine with lower sampling rates and simpler computation, which is the subject of this invention.

The Current Sensor and its use are not particularly limited. In preferred embodiments, the sensor operates to observe/measure/determine the electrical property of the asset over time (e.g. every second, minute, hour or any division/combination thereof etc.) and provide/transmit an indication of the observations to the programmed circuitry/module in a real or delayed timeframe. The individual observations/measurements/determinations preferably occur over a short duration of time (e.g. 0.1 to 5.0 seconds, such as 0.5 seconds) and can be a single measurement from the sensor and/or a combination or average of several measurements, for example sensor measurement which occur at a sampling rate between 100 Hz to 500,000 Hz; for example between 200 to 1000 Hz, between 900 to 1200 Hz, or more preferably between 950 and 1050 Hz, such as 1000 Hz to 1020 Hz.

### Contextual Methods

The model for determining the usage state of an asset has been described above using the signal from a Current Sensor associated with an asset. As described above, the main challenge has been to develop a model without knowing ground truth. However, as mentioned above, many organizations will use a reservation system to allow users to reserve time on an asset. The information contained in such a reservation system can provide increased confidence in the true state of an asset. If this information is then used to train the model, the model's ability to correctly determine the usage state of an asset will improve.

As mentioned above, these reservation systems allow a user to book a specified time to use a specified asset. The asset may be identified with a unique name or alphanumeric identifier (for example, a number, name, or some other alphanumeric identifier). In addition, the asset type may also be indicated (for example, HPLC, mass spectrometer, centrifuge, fume hood, balance, liquid handler, pH meter, incubator, etc).

When a Current Sensor is coupled with an asset and that asset is also booked using the reservation system, then there is additional information that a classification algorithm can use to improve its ability to correctly identify when an asset is in one of the states to be identified. This is because when an asset has been booked for use, there is a higher probability that the signal detected by the Current Sensor is due to the asset being in the “In Use” state during the time window in which it was reserved. As is the case with human operators, the exact start and stop times may fall slightly outside or inside of the time window that the asset was booked for. Regardless, using the reservation times from the reservation system to define a time window within which the asset is deemed to be in the “In Use” state can improve the classification algorithm. One ordinary skilled in the art of machine learning will recognize that this is a method of supervised learning.

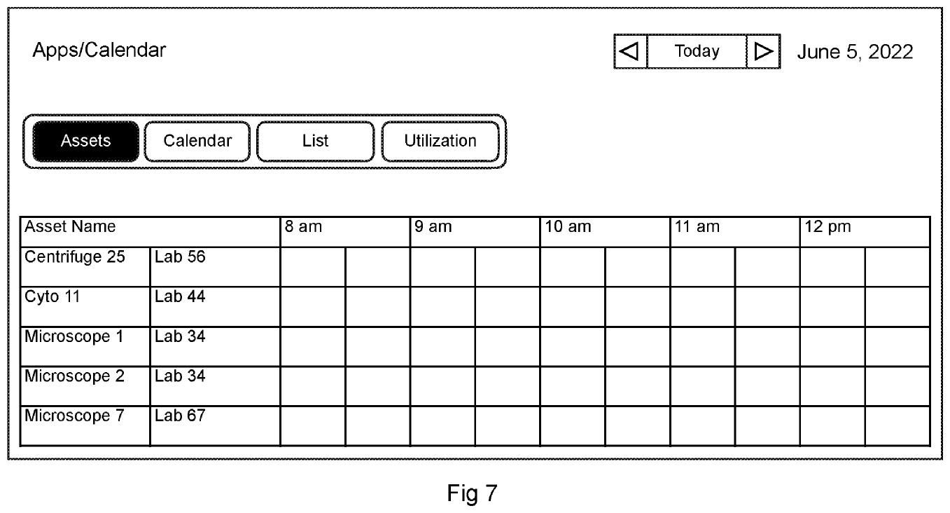

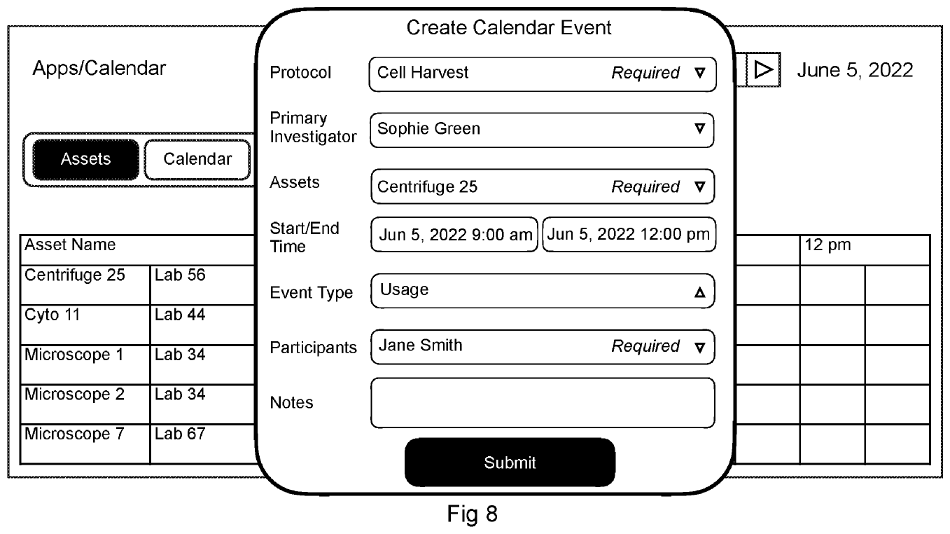

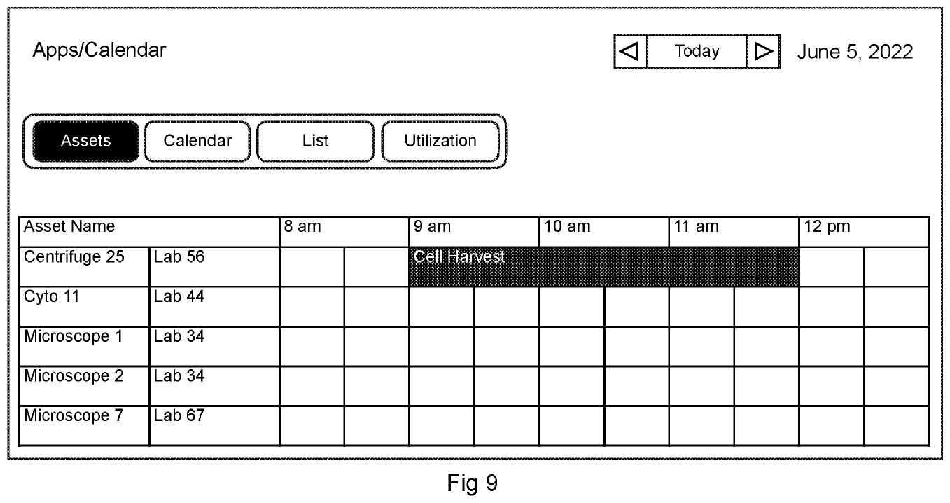

FIGS. 7-9 show examples of a reservation system:
FIG. 7 shows a graphical user interface that lists several rows of assets that are available for reserving.
FIG. 8 shows a pop-up window that allows a user to create a calendar event to reserve an asset for use. In this example, a centrifuge (named “Centrifuge 25”) is being reserved from 9 am to 12 pm on Jun. 5, 2022 for use in a protocol called “Cell Harvest”.
FIG. 9 shows the reservation event in the calendar.

Thus, when a model is being created to determine the different states for this asset (named “Centrifuge 25”), the Current Sensor signal during the time reserved may be labeled as In Use when training a supervised machine learning system. One ordinary skilled in the art will recognize that the exact start and stop times for the In Use state may not exactly correspond to the times booked in the reservation calendar, but that these times define an approximate window where there is a higher likelihood that the asset (“Centrifuge 25”) was in the In Use state.

Another method of using time-based cues to infer and/or estimate ground truth is to take into account the natural working times and cycles that occur. One ordinarily skilled in the art will recognize that there are times of the day and days of the week that users are either more likely to be using an asset (“natural in-use windows”) or less likely to be using an asset (“natural non-use windows”). For example:
Natural in-use windows may be during traditional working hours of a weekday, such as between 8 am-6 pm on a Monday, Tuesday, Wednesday, Thursday, and/or Friday.
Natural non-use windows may be during traditional non-working hours of a weekday, such as:
   between 6 pm Monday-8 am Tuesday
   between 6 pm Tuesday-8 am Wednesday
   between 6 pm Wednesday-8 am Thursday
   between 6 pm Thursday-8 am Friday
   between 6 pm Friday-8 am Saturday
   Natural non-use windows are during weekends, such as on a Saturday and/or Sunday.

Natural non-use windows are during holidays, such as Christmas and other days traditionally observed as non-working holiday days.

Thus, this additional information can be used to further train a machine learning model as follows:
Measure current signals from an asset for a period of time that encompasses at least one natural in-use window and one natural non-use window, but preferably several natural in-use windows and several non-use windows, such as one full weekday, and/or several full weekdays, and/or one full week, and/or two or more full weeks.
For supervised methods, start training a machine learning model by automatically labeling Current Sensor signals measured during naturally in-use windows as “In Use” state and/or Current Sensor signals measured during naturally non-use windows as “Idle” or “Off” states
For unsupervised methods, tune the model to minimize the occurrence of “In-Use” state predictions during non-use windows and/or “Idle” or “Off” states during in-use windows.

As more and more data is collected that spans across multiple natural in-use windows and natural non-use windows, the machine learning model is expected to improve its classification of the different usage states of an asset. This is because the Current Signal readings are expected to have different characteristics statistically speaking during natural in-use windows and natural non-use windows and providing this additional context to a machine learning model gives it additional information for training purposes.

### ADDITIONAL EMBODIMENTS AND DEFINITIONS

In preferred embodiments, the present invention provides a system (and methods of using the system) for determining availability of an asset. The system includes a sensor positioned to determine an electrical property of the asset and a module and/or programmed circuitry having hardware and instructions for performing the following steps to determine availability of the asset.

The module/circuitry/hardware as described and referred to herein are not particularly limited and are well known to those skilled in the art. In particularly preferred embodiments, the module/circuitry/hardware includes any or all of the following types of equipment: computer, processor, storage, memory, audible or visual display, wired or wireless communication devices (e.g. such as those in communication with any of the other components and/or the sensor etc.). The module/circuitry/hardware may be resident in or near an asset and/or may be server or cloud based.

The module/circuitry/hardware are programmed with instructions for performing any of the methods herein described.

In a first step a data set is obtained which contains readings/measurements from the sensor over a period of time as described above. As an optional precursor to the first step an asset and/or sensor is/are provided and the sensor is associated with said asset to determine an electrical property of the asset as described. The electrical property determined by the sensor is not particularly limited but preferably is related to the electrical and/or power consumption of the asset. In preferred embodiments, the property is selected from the group consisting of: current, inductance, voltage, electrical field, magnetic field, and heat etc.

In a second step ranges of sensor readings are determined by performing statistical analysis of the data set obtained in step one. As noted above, this may occur via the statistical analysis approach of clustering. The statistical analysis of clustering can be performed by clustering readings from the sensor obtained in step (ii) into groupings of similar readings of the sensor.

In a third step, asset operating state values can be assigned to the ranges of sensor readings determined in the second step. The asset operating values can be selected from the group consisting of: on, off, idle, and indefinite-sensor offline. Different or additional values can be determined and assigned as desired or required by the end user.

As noted herein, the step of assigning asset operating values to the ranges of sensor readings from the sensor can be performed with the use/aid/assumption/assignment of a ground truth as herein described. The ground truth can be obtained by comparing the sensor reading and a time stamp of the sensor reading obtained in the first step with an asset reservation system. It is contemplated that this can be done by comparing the sensor reading and an associated time stamp with an asset reservation or management system which indicates whether the asset is scheduled to be in use or out of use at/near the timestamp of the sensor reading. If the asset is scheduled to be in use at or near the timestamp of the sensor reading, it can be assumed that the asset is in use and the sensor reading and asset operating value can be assigned an On and/or In Use value. Similarly, if the asset is scheduled to be out of use at or near the timestamp of the sensor reading, it can be assumed that the asset is out of use and the sensor reading and asset operating value can be assigned an Idle/Off/Out of Use value. If no readings are obtained or obtainable from the sensor it might be assumed that the sensor is offline and assigned an indefinite and/or offline status etc.

In additional preferred embodiments, the ground truth can be obtained by assuming that readings from the sensor which occur at certain predefined times equate with certain operating states of the asset. For example, a sensor reading and associated timestamp at certain predefined times (e.g. between 11 pm-4 am and/or on the weekend) is a time when the asset should be idle or off. In such an instance the ground truth can be determined/ascertained/assumed that the asset is in an idle or off state.

In additional steps a further reading from the sensor is obtained and the operating state of the asset can be determined by comparing the further reading from the sensor to the asset operating state values assigned in step (iii), thereby determining the availability of the asset. This can be done by comparing the further sensor reading (e.g. via the use of a look up chart or graphical representation) to the clustered data to determine if the further sensor reading is comparable or falls within the defined cluster ranges etc. Depending on this comparison, the operating state of the asset at about the time (e.g. current time) of the further sensor reading can be determined.

In additional preferred embodiments, instruction further comprises those to perform the step of communicating the operating state of the asset to a user (for example via a message using an audible or visual display). This can be done via a visual or audible message such as a visual display on an asset reservation/management system display or dashboard.

In further preferred embodiments, the present invention provides data files, audible or visual displays, API calls, apparatuses for controlling/coordinating process flows, comprising circuitry programmed with instructions for performing the steps outlined in any of methods described herein, including providing audible or visual messaging (such as a computer display/speaker) to a user.

The present invention further provides a printed set of instructions comprising instructions AND/OR a computer, software package, a module and/or a node programed with logic and/or instructions for performing any and/or all steps of performable by a computer processor comprising instructions to perform any and all of the steps of any method described herein. For example the present invention provides: a method of training a machine learning system comprising any combination of steps recited herein; a data file comprising assigned asset operating values obtained by any or all of the steps described herein; an audible and/or visual display comprising a message indicating availability of an asset, wherein availability of the asset is determined as described herein; a module or hardware comprising programmed instructions for determining availability of an asset, comprising any combination of steps described herein, etc.

Any external reference mentioned herein, including for example websites, articles, reference books, textbooks, granted patents, and patent applications are incorporated in their entirety herein by reference for all purposes.

The present invention includes concepts relating to asset management, record keeping, calendaring, data census and/or entry, data file types, and storage methods. Any of the systems, concepts, file, methods, and/or concepts herein described can be used with data and/or asset management systems. In some embodiments these can include electronic and/or empirical data management systems (EDMSs) (e.g. those optionally containing a calendaring and/or asset management system) and specific uses of such systems. Empirical data management systems (EDMS) can include Laboratory Information Management System (LIMS), Scientific Data Management System (SDMS), Electronic Laboratory Notebook (ELN), and the like.

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments. In addition, it is to be understood that the described element(s) may be combined in any suitable manner in the various embodiments.

Numerical values in the specification and claims of this application reflect average values for a composition. Furthermore, unless indicated to the contrary, the numerical values should be understood to include numerical values which are the same when reduced to the same number of significant figures and numerical values which differ from the stated value by less than the experimental error of conventional measurement technique of the type described in the present application to determine the value.

1-25. (canceled)

26. A system for determining availability of an asset, the system comprising:
a sensor positioned to determine an electrical property of the asset; and
a module and/or programmed circuitry comprising hardware and instructions for performing the following steps to determine availability of the asset:
(i) obtaining a data set comprising readings from the sensor over a period of time;
(ii) determining ranges of sensor readings by performing statistical analysis of the data set obtained in step (i),
(iii) assigning asset operating state values to the ranges of sensor readings determined in step (ii);
(iv) obtaining a further reading from the sensor; and
(v) determining the operating state of the asset by comparing the further reading from the sensor obtained in step (iv) to the asset operating state values assigned in step (iii), thereby determining the availability of the asset.

27. The system of claim 26, wherein step (ii) is performed by the statistical analysis of clustering, for example, wherein the statistical analysis of clustering is performed by clustering readings from the sensor obtained in step (i) into groupings of similar readings of the sensor.

28. The system of claim 26, wherein the asset operating values are selected from the group consisting of: “on”, “off”, “idle”, and “indefinite-sensor offline”.

29. The system of claim 26, wherein the step of (iii) assigning asset operating values to the ranges of sensor readings from the sensor is performed using a ground truth, wherein the ground truth is obtained by comparing the sensor reading and a time stamp of the sensor reading obtained in step (i) with an asset reservation and/or management system, or wherein the ground truth is obtained by assuming that readings from the sensor which occur at certain predefined times equate with certain operating states of the asset (for example a sensor reading and associated timestamp between 11 pm-4 am and/or on the weekend is at a time when the asset is idle or off)

30. The system of claim 26, wherein the electrical property determined by the sensor is related to the electrical and/or power consumption of the asset and is selected from the group consisting of: current, inductance, voltage, electrical field, magnetic field, and heat etc,
wherein the asset comprises a power cord and the sensor is associated with the power cord, and
wherein the system further comprises the asset wherein the asset is optionally selected from the group consisting of: laboratory or manufacturing equipment.

31. The system of claim 26, further comprising the step of communicating the operating state of the asset to a user (for example via a message using an audible or visual display).

32. A method for determining availability of an asset:
(i) positioning a sensor to determine an electrical property of the asset;
(ii) using the asset over a period of time and obtaining a data set comprising readings from the sensor over the period of time;
(iii) determining ranges of sensor readings by performing statistical analysis of the data set obtained in step (ii),
(iv) assigning asset operating state values to the ranges of sensor readings determined in step (iii);
(v) obtaining a further reading from the sensor; and
(vi) determining the operating state of the asset by comparing the further reading from the sensor obtained in step (v) to the asset operating state values assigned in step (iv),
thereby determining the availability of an asset.

33. The method of claim 32, wherein step (iii) is performed by the statistical analysis of clustering, for example, wherein the statistical analysis of clustering is performed by clustering readings from the sensor obtained in step (ii) into groupings of similar readings of the sensor.

34. The method of claim 32, wherein the asset operating values are selected from the group consisting of: on, off, idle, and sensor offline.

35. The method of claim 32, wherein the step of (iv) assigning asset operating values to the ranges of sensor readings from the sensor is performed using a ground truth,
wherein the ground truth is obtained by comparing the sensor reading and a time stamp of the sensor reading obtained in step (ii) with an asset reservation and/or management system or
wherein the ground truth is obtained by assuming that readings from the sensor which occur at certain predefined times equate with certain operating states of the asset (for example a sensor reading and associated timestamp between 11 pm-4 am and/or on the weekend is at a time when the asset is idle or off)

36. The method of claim 32, wherein the electrical property determined by the sensor is related to the electrical and/or power consumption of the asset and is selected from the group consisting of: current, inductance, voltage, electrical field, magnetic field, and heat etc,
wherein the asset comprises a power cord and the sensor is associated with the power cord, and
wherein the asset is laboratory or manufacturing equipment.

37. The method of claim 32, further comprising the step of communicating the operating state of the asset to a user (for example via a message using an audible or visual display).

38. A method of training a machine learning system comprising any combination of steps recited in claim 32.

39. A data file comprising assigned asset operating values obtained by the method of any one of claim 32.

40. An audible and/or visual display comprising a message indicating availability of an asset, wherein availability of the asset is determined by the method of claim 32.

41. A module or hardware comprising programmed instructions for determining availability of an asset, comprising any combination of steps of the method of claim 32.
